(19) United States
(12) Patent Application Publication
(10) Pub. No.: US 2021/0121322 A1
(43) Pub. Date: Apr. 29, 2021

(54) EYE DROPPER GRIP WITH MIRROR

(71) Applicant: CLARISSA PRIETO, CLEVELAND, TX (US)

(72) Inventor: CLARISSA PRIETO, CLEVELAND, TX (US)

(21) Appl. No.: 17/080,591

(22) Filed: Oct. 26, 2020

Related U.S. Application Data
(60) Provisional application No. 62/925,962, filed on Oct. 25, 2019.

(51) Int. Cl. A61F 9/00; A47G 1/02; F16B 2/22; F16M 13/02

(52) U.S. Cl. CPC A61F 9/0008; F16M 13/022; F16B 2/22; A47G 1/02

(57) ABSTRACT

The dropper mount assembly includes an angled mirror and a clip that mounts to an eye drop bottle. The angled mirror allows the user to gain proper alignment by viewing a reflection of the expected trajectory of the medicinal eye drop to the eye of the user. The clip is configured to receive eye drop bottles having a circular or oval cross section and is capable of either being mounted from an axis end of the eye drop bottle or from a side of the eye drop bottle. The clip has a set of prongs that provide a number of features, including providing spring tension against the eye dropper bottle, are permitted to receive eye dropper bottles of variable sizes, and serve as a grip and leverage that assists the user in squeezing the eye drop bottle when the user contracts the prongs together.

### CROSS REFERENCE TO RELATED APPLICATIONS

This application claims the benefit of an earlier filing date and right of priority to U.S. Provisional Application No. 62925962, filed 25 Oct. 2019, the contents of which is incorporated by reference herein in its entirety.

### BACKGROUND OF THE INVENTION

### 1. Field of the Invention

The present application relates to medicine droppers, and more particularly to eye droppers equipped with a reflector for guiding placement of eye drops into an eye of a user.

### 2. Description of Related Art

In general, medicinal eye drops are dispensed from a dropper bottle while the dropper bottle is in an inverted position located above an eye of a user such that the eye drops exit from a nozzle of the dropper bottle and fall into the eye of the user while the user is looking up. To dispense the medicinal eye drops, the user gently squeezes the sides of the dropper bottle to increase internal pressure of the dropper bottle, which subsequently forces the medicinal liquid to be expelled from the nozzle of the bottle.

Correct positioning of the dropper bottle while applying the medicinal eye drops can be difficult endeavor for a user for a variety of reasons: the user may be inexperienced at applying eye drops, may feel uncomfortable and hesitant about the sensation of dropping liquids into the eye, or may have a fine motor skill disability that makes positioning or squeezing of the dropper bottle difficult for the user. To overcome these difficulties, a variety of eye dropper guides have been proposed that assist the user to position the bottle. For example, an eye dropper guide having an angled mirror that couples to the dropper bottle permits the user to view the reflection of the nozzle of the dropper bottle and the eye of the user in order to ensure proper alignment of the expected trajectory of the medicinal eye drop to the eye of the user.

However, the proposed solutions have several disadvantages. Some proposed solutions describe a ring mount that couples to the dropper bottle to either the squeezable body of the dropper bottle or the neck portion of the dropper bottle, however, this solution is limited to only a particular bottle shape and size. Furthermore, the ring mount requires the user to slide the ring mount over an end of the dropper bottle, which may be difficult for users who have difficulty with fine motor skills. In some instances, the dropper bottle may be difficult to squeeze to expel the medicinal drops for a user that has weakened hand strength. In this situation, the previously mentioned solutions offer no relief since the ring mount is static and inflexible, i.e., the ring mount does not assist in squeezing the dropper bottle.

Although strides have been made to assist in applying eye drops from a dropper bottle through a variety of eye dropper guides, some shortcomings remain. It is desired to make an eye dropper guide that accepts dropper bottles of different sizes and shapes, that easily permits the user to mount the dropper guide from a side of the bottle (i.e., perpendicular to a bottle axis), and assists the user in squeezing the bottle to discharge the medicinal drops.

### BRIEF SUMMARY OF THE INVENTION

It is an object of the present application to make an eye dropper guide that accepts dropper bottles of different sizes and shapes, that easily permits the user to mount the dropper guide from a side of the bottle (i.e., perpendicular to a bottle axis), and assists the user in squeezing the bottle to discharge the medicinal drops.

The dropper mount assembly of the present application is configured to include a mirror and a clip associated therewith configured to removably clip onto and eye drop bottle from the side. The clip includes a plurality of prongs that provide spring tension against the eye dropper and to provide grip and leverage to user so as to minimize the force needed from the user to compress the eye dropper.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a dropper mount assembly, in accordance with an embodiment of the present application.

FIG. 2 is a side view of the dropper mount assembly, in accordance with the embodiment of the present application.

FIG. 3 is a front view of the dropper mount assembly, in accordance with the embodiment of the present application.

FIG. 4 is an exemplary use of the dropper mount assembly coupled with an eye drop bottle having an oval cross section, in accordance with the embodiment of the present application.

FIG. 5 is an exemplary use of the dropper mount assembly coupled with an eye drop bottle having a circular cross section, in accordance with an embodiment of the present application.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The embodiments and method of the present application is illustrated in the associated drawings. In particular, the system of the present invention is a dropper mount assembly having an angled mirror and a U-shaped clip that mounts to an eye drop bottle. The angled mirror allows the user to view the reflection of the nozzle of the dropper bottle and the eye of the user in order to ensure proper alignment of the expected trajectory of the medicinal eye drop to the eye of the user. The U-shaped clip is configured to receive eye drop bottles having a circular or oval cross section and is capable of either being mounted from an axis end of the eye drop bottle or from a side of the eye drop bottle. Furthermore, the U-shaped clip has a set of prongs that provide a number of features: 1) the prongs provide spring tension against the eye dropper bottle that assist in coupling the dropper mount assembly to the eye dropper bottle, 2) the prongs are permitted to receive eye dropper bottles of variable sizes by virtue of the provided spring tension, and 3) the prongs serves as a grip and leverage that assists the user in squeezing the eye drop bottle when the user contracts the prongs together. Additional features and functions are illustrated and discussed below.

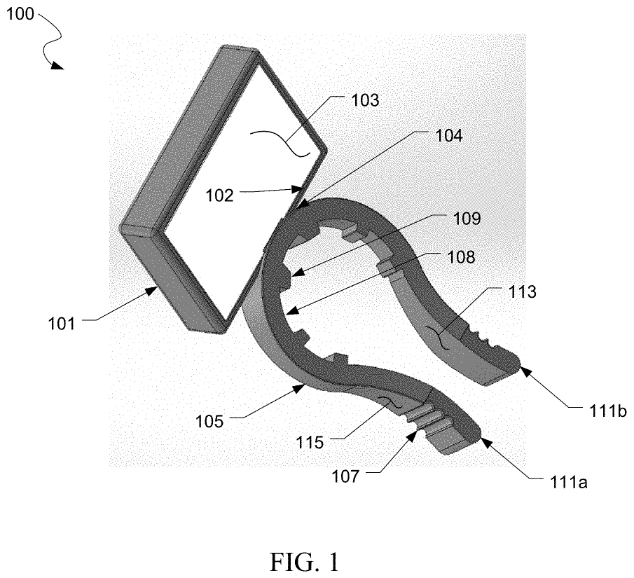

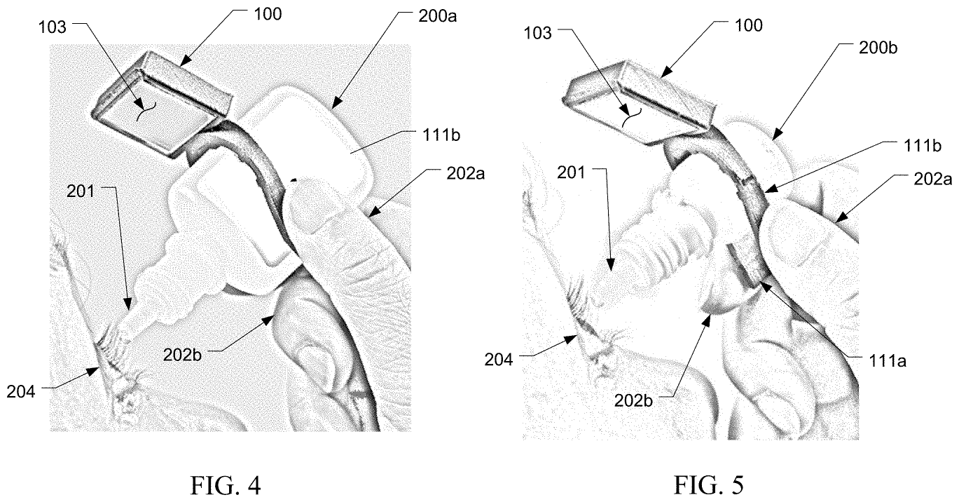

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise. Likewise, reference characters identify corresponding or similar elements in form and function through the several views. FIGS. 1-3 illustrate assorted views of a dropper mount assembly. FIGS. 4-5 illustrate exemplary uses of the dropper mount assembly with dropper bottles having circular and oval cross sections.

Referring now to FIGS. 1-3, a views of dropper mount assembly 100 is illustrated to show in accordance with an embodiment of the present invention. In FIG. 1, dropper mount assembly 100 includes, but is not limited to, a base plate 101, a mirror 103, and a U-shaped clip 105. Base plate 101 is coupled to clip 105 and provides relative motion between the two. Base plate 101 can therefore optionally pivot and/or rotate relative to clip 105. This allows a user to angle base plate 101 to a preferred position for the user.

In this embodiment, base plate 101 is a square or rectangular plate configured to receive mirror 103. Mirror 103 is coupled to a surface of base plate 101 and is used to reflect an image of the user to allow the user to see and orient the dropper to the eye. Mirror 103 is adhered planarly to the base plate 101. In this embodiment, mirror 103 can be, but is not limited to, a polished metal mirror, a polished metal mirror having a glass cover, a polished plastic mirror, or a polished plastic mirror having a glass cover.

In this embodiment, the U-shaped clip 105 is a clip that is configured to receive dropper bottles having either a circular or oval cross section. Clip 105 has a set of prongs that oppose each other (i.e., prong 111a and 111b). Each prong is curved, coupling together at a proximate end (i.e., end 104) and are separated at a distal end. Prongs 111a/111b are planar to each other and are configured to flex relative to one another at the distal end such that the distance between the prongs can change as pressure is applied to the prongs.

In this embodiment, clip 105 is made of a material that permits prongs 111a and 111b to be springly compressed and expanded relative of each other. For example, clip 105 can be made of metal or plastic. In this embodiment, an external face (i.e., face 115) of each prong is texturized to form a grip for a user. For example, in FIG. 1, grip ridges 107 are on prongs 111a and 111b located on face 115. In this embodiment, clip 105 has a partially circular chamber (i.e., chamber 108) that is located proximate to end 104 that receives a dropper bottle having either a circular or oval cross section. Furthermore, clip 105 has a plurality of coupling ridges (i.e., coupling ridges 109) located on face 113 along chamber 108, wherein coupling ridges 109 minimize surface area contact between clip 105 and a dropper bottle in order to permit clip 105 to slide along an axis of the dropper bottle with reduced friction should the user need to reposition dropper mount assembly 100. Ridges 109 make it easier to configure to any shape and size of an eye drop bottle therefore more or less pressure can be added to that specific bottle for the applicator to fit. It is important to remember that base plate 101 may be integrally coupled with clip 105 adjacent to end 104 either integrally or in a manner that allows movement.

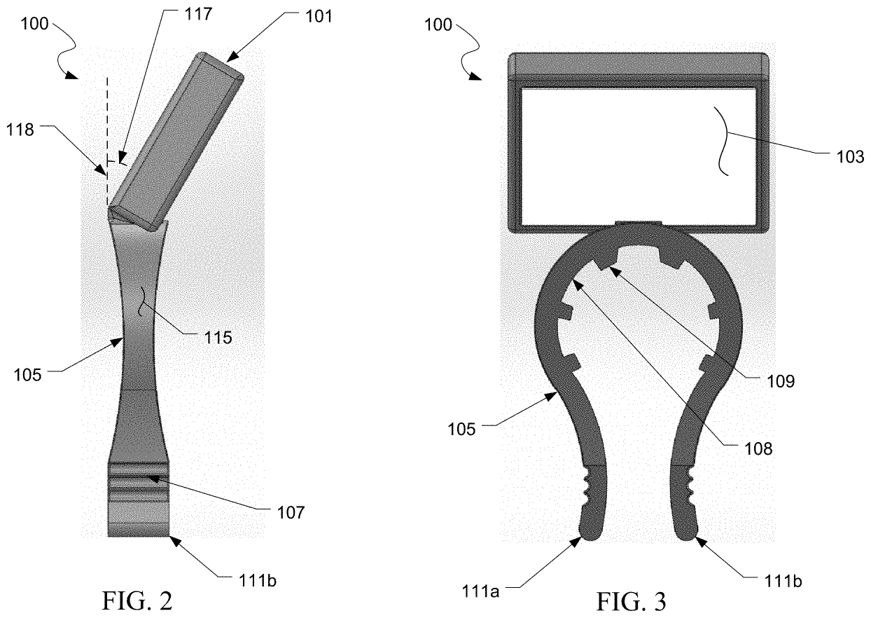

With respect to FIG. 2 in the drawings, a side view of dropper mount assembly 100 is illustrated in accordance with the embodiment of the present application. In this embodiment, base plate 101 is shown in an angled orientation relative to the body of clip 105. For example, in reference to FIG. 2, base plate 101 has angle 117 relative to clip 105, wherein the angle of angle 117 is 30 degrees relative to a normal vector (i.e., line 118) of an axis of chamber 108.

Now in reference to FIG. 3, a front view of dropper mount assembly 100 is illustrated in accordance with the embodiment of the present invention.

Referring now also to FIG. 4, an exemplary use of dropper mount assembly 100 coupled with an eye drop bottle having an oval cross section is illustrated in accordance with the embodiment of the present invention.

In FIG. 4, dropper mount assembly 100 is coupled with an eye drop bottle (i.e., dropper bottle 200a) by sliding dropper bottle 200a between prongs 111a and 111b (prong 111a is hidden from view) until dropper bottle 200a is positioned within chamber 108. Alternatively, the user couples dropper mount assembly 100 to dropper bottle 200a by sliding an axial end of the eye dropper bottle into chamber 108 of dropper mount assembly 100.

In using dropper mount assembly 100, the user positions the coupled dropper mount assembly 100 and dropper bottle 200a overhead, inverts bottle 200a such that a nozzle of the dropper bottle (i.e., nozzle 201) is directed toward the user, and aligns nozzle 201 with an afflicted eye of the user designated to receive medicinal eye drops (i.e., eye 204) by peering into mirror 103 with eye 204 and using mirror 103 to guide positioning of nozzle 201 over eye 204 of the user. The user then positions a set of fingers of the user (i.e., fingers 202a and 202b) on prongs 111a and 111b respectfully. To produce eye drops from bottle 200a, the user squeezes prongs 111a and 111b together using fingers 202a and 202b, which in turn compresses the body of bottle 200a and subsequently discharges eye drops from nozzle 201.

In this example, by positioning dropper bottle 200a within chamber 108, prongs 111a and 111b are expanded relative to each other and are, as a result of the expansion, under spring tension against dropper bottle 200a. Furthermore, as a result of the expansion between prongs 111a and 111b, prongs 111a and 111b become splayed outward and serve as levers for the user in compressing dropper bottle 200a, thereby assisting the user in compressing dropper bottle 200a.

Now in reference to FIG. 5, an exemplary use of dropper mount assembly 100 coupled with an eye drop bottle having a circular cross section is illustrated in accordance with the embodiment of the present invention.

In FIG. 5, eye drop bottle (i.e., dropper bottle 200b) has a circular cross section. In this example, the method of use of dropper mount assembly 100 is in similar form and function as that taught by FIG. 4.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A dropper mount assembly, comprising:
a base plate;
a pair of prongs coupled to the base plate; and
a mirror coupled to a face of the base plate;
wherein the mirror is configured to adjust in position relative to the pair of prongs.

2. The assembly of claim 1, wherein the base plate is rectangular.

3. The assembly of claim 1, wherein the pair of prongs are coupled together at a proximate end; the base plate coupled to the pair of prongs at the proximate end.

4. The assembly of claim 3, wherein the pair of prongs are separated at a distal end of the pair of prongs.

5. The assembly of claim 1, wherein at least one of the pair of prongs includes a texturized grip at a distal end.

6. The assembly of claim 1, wherein the pair of prongs are configured to flex at a proximate end so as to change a distance between the pair of prongs at a distal end of the pair of prongs.

7. The assembly of claim 1, wherein the pair of prongs include coupling ridges.

8. The assembly of claim 7, wherein the coupling ridges extend inward along a face of the pair of prongs, the coupling ridges partially face each other.

9. The assembly of claim 1, wherein the base plate is configured to pivot relative to the pair of prongs.

10. The assembly of claim 1, wherein the base plate is configured to rotate relative to the pair of prongs.

11. A dropper mount assembly, comprising:
a base plate;
a pair of prongs movably coupled to the base plate, the pair of prongs being joined at a proximate end, the pair of prongs being planar to each other and curved so as to be separated at a distal end, the curve creates a void between the distal end and the proximate end; and
a mirror coupled to a face of the base plate;
wherein the mirror is configured to adjust in position relative to the pair of prongs.

12. The assembly of claim 11, wherein the base plate is rectangular.

13. The assembly of claim 11, wherein at least one of the pair of prongs includes a texturized grip at a distal end.

14. The assembly of claim 11, wherein the pair of prongs are configured to flex at a proximate end so as to change a distance between the pair of prongs at a distal end of the pair of prongs.

15. The assembly of claim 11, wherein the pair of prongs include coupling ridges.

16. The assembly of claim 15, wherein the coupling ridges extend inward along a face of the pair of prongs, the coupling ridges partially face each other.

17. The assembly of claim 11, wherein the base plate is configured to pivot relative to the pair of prongs.

18. The assembly of claim 11, wherein the base plate is configured to rotate relative to the pair of prongs.
